(19) United States
(12) Patent Application Publication
(10) Pub. No.: US 2023/0068913 A1
(43) Pub. Date: Mar. 2, 2023

(54) PERSONAL RADIATION PROTECTIVE DEVICE

(71) Applicant: Relentless Innovation Pty Ltd, Shailer Park (AU)

(72) Inventor: Glenn HONEY, Shailer Park (AU)

(73) Assignee: Relentless Innovation Pty Ltd, Shailer Park (AU)

(21) Appl. No.: 17/822,177

(22) Filed: Aug. 25, 2022

(30) Foreign Application Priority Data
Aug. 25, 2021 (AU) 2021221588

(51) Int. Cl. A61B 6/10; G21F 3/02

(52) U.S. Cl. CPC A61B 6/107; G21F 3/02

(57) ABSTRACT

A personal radiation protective device comprising a radiation barrier and an outer covering that covers the radiation barrier; the outer cover made from a material herein one or more edges of the outer covering are welded together.

### CROSS REFERENCE TO RELATED APPLICATIONS

This application claims priority to, and the benefits of Australian Patent Application 2021221588, filed on Aug. 25, 2021, the contents of which are hereby incorporated by reference in their entirety.

### FIELD OF THE INVENTION

This invention relates to a personal radiation protective device. In particular, the invention relates to a personal radiation protective device used to protect individuals who perform imaging procedures such as X-rays, CT scans, nuclear medicine scans and PET scans.

### BACKGROUND OF THE INVENTION

Diagnostic radiology procedures, such as computed tomography (CT) and X-ray, are commonplace in hospitals and medical centres for diagnosing a variety of medical conditions. The downside of diagnostic radiology procedures is persons are exposed to ionising radiation, which is associated with increased risk of malignancy, proportional to the level of exposure. These procedures generally have a very low risk of causing harm to a patient. However, with an increase in the number of procedures and the type of procedures, the risk of harm increases. Similarly, for a practitioner who operates or works adjacent diagnostic radiology equipment there is the potential for increased exposure to ionising radiation.

To reduce the exposure to ionising radiation to both patients and practitioners, personal radiation protective devices have been created. These protective devices include a radiation barrier which absorbs the energy of the radiation to reduce the radiation to a level safe for humans. Traditionally a radiation barrier has been made from lead. However, due to their weight and toxicity, other non-lead materials have been utilised such as tin, antimony, tungsten, bismuth and combinations thereof.

Personal protective devices also include an outer covering which covers the radiation barrier. The outer covering can be made from a variety of different materials such as woven and non-woven nylon and polyurethane. The selection of materials is often based on durability, flexibility, and denier to reduce tearing and fraying while not impeding on the users use of the protective device.

The outer covering is often sown together using pieces of material that are cut to specific shapes depending on the specific use of the personal protection device. Accordingly, stitched seams are often created on the outer covering especially at the edges where seams also assist in preventing fraying. Stitched joins are strong and effective in producing a usable outer covering for a protection device.

Unfortunately, stitched joins provide microbial traps where microorganisms (such as bacteria, viruses, moulds, fungi, algae and protozoa) can hide and multiply even when a personal protective device is thoroughly cleaned with disinfectant. This can lead to poor infection control in a medical environment, such as a hospital, where protection devices are used by different people in different areas. Furthermore, stitched seams can be contaminated with bodily fluids such as blood, urine and faeces which are also difficult to clean. This can often lead to the protection device being discarded which is expensive.

### OBJECT OF THE INVENTION

It is an object of the invention to overcome and/or alleviate the abovementioned problems and/or provide the consumer with a useful or commercial choice.

### SUMMARY OF THE INVENTION

In one form, although not necessarily the only or broadest form, the invention resides in a personal radiation protective device comprising:
a radiation barrier; and
an outer covering that covers the radiation barrier; the outer cover made from a material
wherein one or more edges of the outer covering are welded together.

Normally, the outer covering is made by two or more pieces of material. Preferably, any join on the outer covering which joins two or more pieces of material together is welded. A join includes the edges of the outer covering.

The radiation barrier may be made from any suitable type of radiation shield material. The radiation barrier may be made from lead based or non-lead based material. The non-lead based material may include tin and/or antimony and/or tungsten and/or bismuth or combination thereof.

The outer covering may be between 0.1 mm and 2 mm thick.

The weld may be a radio frequency weld, dielectric weld, high frequency weld, electrical impulse weld or heat weld.

The outer covering may be made from any type of cover material that is able to be welded. Preferably, the cover material is a textile or fabric. The textile or fabric may be a woven or non-woven material. The textile or fabric may be a polymer or a plastic. Normally the textile is polar molecular plastic. Suitable polymers may include polyvinyl chloride coated fabrics, polyvinyl chloride foam, chlorinated polyvinyl chloride, nylon, rayon, polyester, acrylic, spandex, olefin, polyolefin, neoprene, lycra, cellulose acetate, ethylene vinyl acetate, thermoplastic polyurethane, thermoplastic polyurethane coated fabrics, thermoplastic elastomers, chlorinate polyethylene, polyvinylidene chloride or polyethylene terephthalate.

The outer covering may include an antimicrobial protective coating such as quaternary ammonium compounds, silver ions, zinc antimicrobials and copper antimicrobials, or other antimicrobial reagents.

The personal radiation protective device may include a closure mechanism. The closure mechanism may be attached to the outside of the outer covering. Preferable the closure mechanism is at least partially welded to the outer covering. The closure mechanism may be a belt. The belt may include a clasp, clip, buckle or other type of fastener.

The closure mechanism may be located inside the outer covering. The closure mechanism may include one or more magnets. Typically, there are at least two magnets. The magnets may be located within an over lapping portion of the outer covering. The magnets may be positioned laterally within the outer covering to provide closure for larger and smaller people.

Indicia may be located on the outer covering to indicate a safe level of overlap of the outer covering.

The personal radiation protective device may include a digital readable element. The digital readable element may be used to track the usage of the personal radiation protective device. The digital readable element may be an RFID tag. The RFID tag may be located on or inside the outer covering. Alternatively or additionally, a barcode tag may be located on the outer covering. The barcode tag may be used to track the usage of the personal radiation protective device.

In another form, the invention may reside in a method of producing a personal radiation protective device comprising the steps of:
cutting an outer covering to a desired dimension;
cutting or forming a radiation barrier to a desired dimension; and
welding the edges of the outer covering together.

Further features of the invention will become apparent from the detailed description below.

### BRIEF DESCRIPTION OF THE DRAWINGS

Embodiments of the invention will be described, by way of example only, with reference to the accompanying figures in which:

FIG. 1 is a top view of a multipurpose drape according to a first embodiment of the invention;

FIG. 2 is a top view of a multipurpose drape according to a second embodiment of the invention;

FIG. 3 is a top view of an etoposide and cisplatin (EP) shield according to a third embodiment of the invention;

FIG. 4 is a top view of a biliary split shield according to a fourth embodiment of the invention;

FIG. 5 is a top view of a femoral entry angiography drape according to a fifth embodiment of the invention;

FIG. 6 is a top view of a peripheral shield according to a sixth embodiment of the invention;

FIG. 7 is a front view of an apron according to a seventh embodiment of the invention;

FIG. 8 is a front view of an apron according to a eighth embodiment of the invention;

FIG. 9 is a front view of an apron according to a ninth embodiment of the invention;

FIG. 10 is a perspective view of an apron according to a tenth embodiment of the invention;

FIG. 11 is a perspective view of an apron according to an eleventh embodiment of the invention;

FIG. 12 is a perspective view of an apron according to a twelfth embodiment of the invention;

FIG. 13 is a perspective view of an apron according to a thirteenth embodiment of the invention;

FIG. 14 is a perspective view of an apron according to a fourteenth embodiment of the invention; and

FIG. 15 is a perspective view of an apron according to a fifteenth embodiment of the invention.

### DETAILED DESCRIPTION OF THE PREFERRED EMBODIMENTS

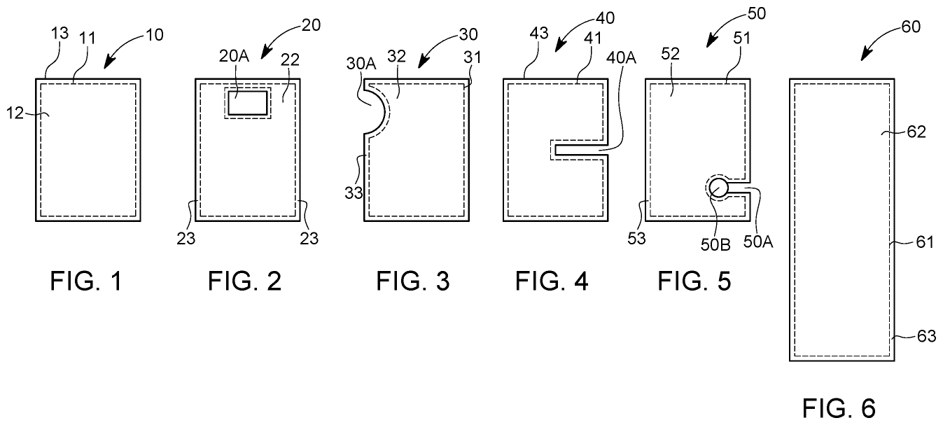

FIG. 1 shows a personal protective radiation device in the form of a multipurpose drape 10 that can be used for a variety of purposes for patients when conducting X-rays or CT scans. The multipurpose drape 10 includes a flexible internal radiation barrier 11 surrounded by a textile outer covering 12. The radiation barrier 10 is made from a sheet of a non-lead based, propriety material RadSafe Optima™. It should be appreciated that other radiation barriers may be used that are know in the art. The outer covering 12 is made from two, non-woven nylon sheets which each have a denier of 70. The edges 12 of the outer covering are radio-frequency welded together.

To produce the multipurpose drape 10, the nylon sheets and radiation barrier 11 is cut to desired dimensions. The edges of the nylon sheets overhang the edges of the radiation barrier 11. The overhanging nylon sheets are located together and radio-frequency welded together to complete the multipurpose drape 10.

FIG. 2 shows personal protective radiation device in the form of a further multipurpose drape 20 having a fenestration 20A located adjacent an end of the multipurpose drape 20. The multipurpose drape 20 includes a flexible internal radiation barrier 21 surrounded by a textile outer covering 22. The radiation barrier 21 is made from a sheet of a non-lead based, propriety material RadSafe Optima™. The outer covering 22 is made from two woven nylon sheets which have a denier of 150. The woven nylon sheets are known in the art as Ripstop nylon as it assists in preventing tearing. The edges 23 of the outer covering 21 are radio-frequency welded together including the edges 23 located around the fenestration 20A.

To produce the multipurpose drape 20, the nylon sheets and radiation barrier 21 sheet is cut to desired dimensions. The edges of the nylon sheets overhang the edges of the radiation barrier 21. The overhanging nylon sheets are located together and radio-frequency welded to complete the multipurpose drape 20.

FIG. 3 shows a personal protective radiation device in the form of an EP shield 30 having a scoop 30A located adjacent an end of the EP shield. The EP shield includes a flexible internal radiation barrier 31 surrounded by a textile outer covering 32. The radiation barrier 31 is made from a non-lead based proprietary material. The outer covering 32 is made from two polyvinyl chloride sheets which have a 1 mm thickness. The edges 33 of the outer covering are radio-frequency welded together.

To produce the EP shield 30, the polyvinyl chloride sheets and radiation barrier 31 is cut to desired dimensions. The edges 33 of the polyvinyl chloride sheets overhang the edges of the radiation barrier. The overhanging polyvinyl chloride sheets are located together and welded together using radio-frequency welding to complete the EP shield 30.

FIG. 4 shows personal protective radiation device in the form of a biliary split shield 40. The biliary spit shield 40 includes a flexible internal radiation barrier 41 surrounded by a textile outer covering 42. The radiation barrier 41 is made from a sheet of a non-lead based, propriety material called RadSafe Optima™. The outer covering 42 is made from two polyurethane sheets which have a thickness of 0.75 mm. The edges 43 of the outer covering are radio-frequency welded together including the edges 43 located along a spit 40A in the biliary split shield.

To produce the biliary split shield 40, the polyurethane sheets and radiation barrier 41 is cut to desired dimensions. The edges 43 of the polyurethane sheets overhang the edges of the radiation barrier 41. The overhanging polyurethane sheets are located together and radio-frequency welded to complete the biliary split shield 40.

FIG. 5 shows personal protective radiation device in the form of a femoral entry angiography drape 50. The femoral entry angiography drape 50 includes a flexible internal radiation barrier 51 surrounded by a textile outer covering 52. The radiation barrier 51 is made from a sheet of a non-lead based, propriety material RadSafe Optima™. The outer covering 52 is made from two polyethylene terephthalate sheets which have a thickness of 0.9 mm. The edges 53 of the outer covering are radio-frequency welded together including the edges 53 located along a split 50A and circular fenestration 50B in the femoral entry angiography drape.

To produce the femoral entry angiography drape 50, the polyethylene terephthalate sheets and radiation barrier 51 is cut to desired dimensions. The edges 53 of the polyethylene terephthalate sheets overhang the edges 53 of the radiation barrier 51. The overhanging polyethylene terephthalate sheets are located together and radio-frequency welded to complete the femoral entry angiography drape 50.

FIGS. 6 shows personal protective radiation device in the form of a peripheral shield 60. The peripheral shield 60 includes a flexible internal radiation barrier 61 surrounded by a textile outer covering 62. The radiation barrier is made from a sheet of a non-lead based, propriety material RadSafe Optima™. The outer covering 62 is made from two, non-woven nylon sheets which each have a denier of 300. The edges 63 of the outer covering 62 are heat welded together.

To produce the peripheral shield 60, the nylon sheets and radiation barrier is cut to desired dimensions. The edges 63 of the nylon sheets overhang the edges 63 of the radiation barrier 61. The overhanging nylon sheets are located together and heat welded together to complete the peripheral shield 60.

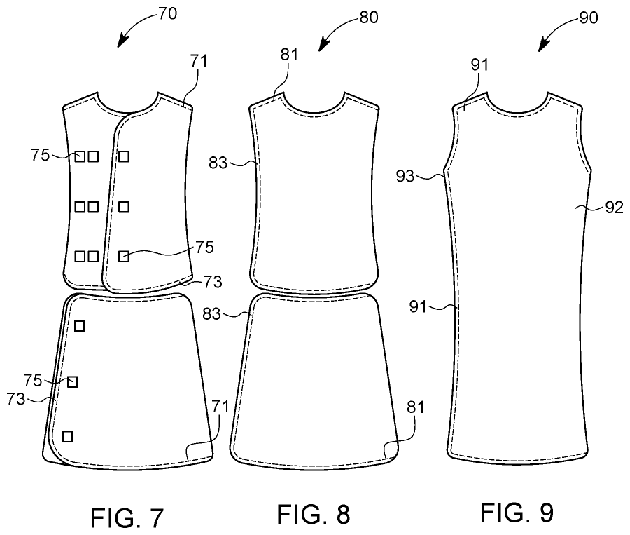

FIG. 7 shows personal protective radiation device in the form of an adjustable two piece apron 70. The apron includes a flexible internal radiation barrier 71 surrounded by a textile outer covering 72. The radiation barrier 61 is made from a sheet of a non-lead based, propriety material RadSafe Optima™. The outer covering 72 is made from two woven nylon sheets which have a denier of 200. The woven nylon sheets are known in the art as Ripstop nylon as it assists in preventing tearing. The edges 73 of the outer covering 72 are radio-frequency welded together.

A number of magnets 75 are located in the outer covering 72 adjacent the over lapping portion of the apron 70. Magnets 75 are located on the inside of a top overlapping portion and the outside of the bottom overlapping portion so that the magnets 75 can attract each other to form a closure mechanism. The magnets 75 are located in a series on the bottom overlapping portion to cater for different sized users. Safety indicia (not shown) is located on the apron to show a minimum amount of overlap required. An RFID tag (not shown) is located within the outer covering.

To produce the apron 70, the nylon sheets and radiation barrier 71 is cut to desired dimensions. The magnets 75 and an RFID tag are fixed to the inside of the nylon sheets in predetermined positions. The edges 73 of the nylon sheets overhang the edges of the radiation barrier 71. The overhanging nylon sheets are located together and radio-frequency welded to complete the two piece apron 70.

FIG. 8 shows a personal protective radiation device in the form of a fixed size two piece apron 80. The two piece apron a flexible internal radiation barrier 81 surrounded by a textile outer covering 82. The radiation barrier 81 is made from a lead sheet. The outer covering 82 is made from two polyvinyl chloride sheets which have a 0.6 mm thickness. The edges 83 of the outer covering 82 are radio-frequency welded together.

To produce the apron 80, the polyvinyl chloride sheets and radiation barrier 81 is cut to desired dimensions. The edges 83 of the polyvinyl chloride sheets overhang the edges of the radiation barrier 81. The overhanging polyvinyl chloride sheets are located together and welded together using radio-frequency welding to complete the two-piece apron 80.

FIG. 9 shows personal protective protection device in the form of a single piece apron 90. The single piece apron includes a flexible internal radiation barrier 91 surrounded by a textile outer covering 92. The radiation barrier 91 is made from a sheet of a non-lead based, propriety material RadSafe Optima™. The outer covering 92 is made from two polyethylene terephthalate sheets which have a thickness of 0.45 mm. The edges 93 of the outer covering 92 are radio-frequency welded together.

To produce the single piece apron 90, the polyurethane sheets and radiation barrier 91 is cut to desired dimensions. The edges 93 of the polyethylene terephthalate sheets overhang the edges of the radiation barrier 91. The overhanging polyethylene terephthalate sheets are located together and radio-frequency welded to complete the single piece apron 100.

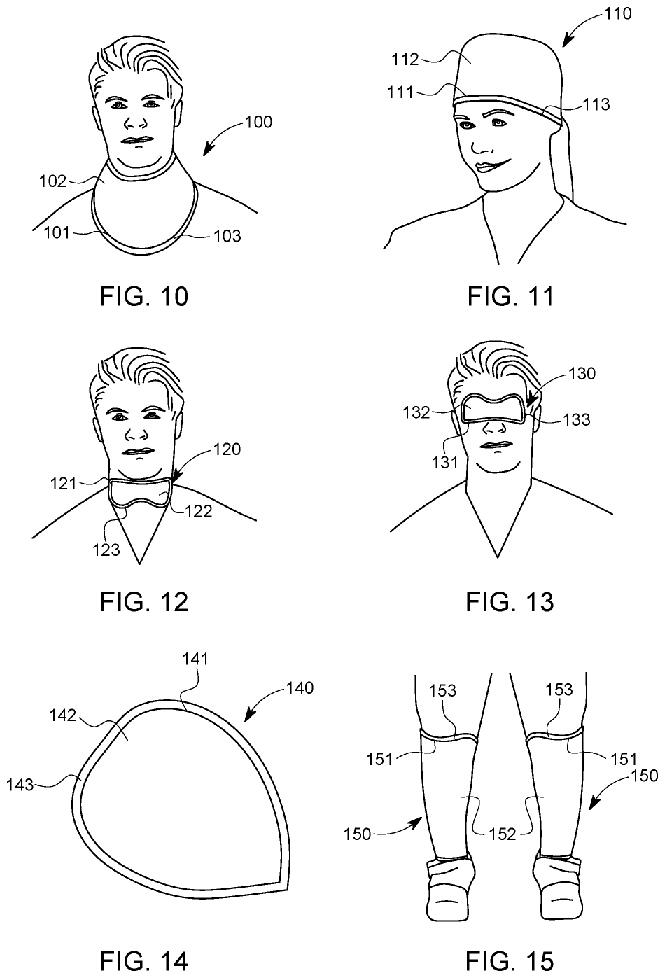

FIG. 10 shows personal protective radiation device in the form of a thyroid collar 100. The thyroid collar 100 includes a flexible internal radiation barrier 101 surrounded by a textile outer covering 102. The radiation barrier 101 is made from a sheet of a non-lead based, propriety material RadSafe Optima™. The outer covering 102 is made from two polyethylene terephthalate sheets which have a thickness of 1.3 mm. The edges 103 of the outer covering 102 are radio-frequency welded together.

To produce the thyroid collar 100, the polyethylene terephthalate sheets and radiation barrier 101 is cut to desired dimensions. The edges 103 of the polyethylene terephthalate sheets overhang the edges 103 of the radiation barrier 101. The overhanging polyethylene terephthalate sheets are located together and radio-frequency welded to complete the thyroid collar 100.

FIG. 11 shows personal protective radiation device in the form of a smart cap 110. The smart cap 110 includes a flexible internal radiation barrier 111 surrounded by a textile outer covering 112. The radiation barrier 111 is made from a sheet of a non-lead based, propriety material RadSafe Optima™. The outer covering 112 is made from two woven nylon sheets which have a denier of 320. The woven nylon sheets are known in the art as Ripstop nylon as it assists in preventing tearing. The edges 113 of the outer covering 112 are radio-frequency welded together.

To produce the smart cap 110, the nylon sheets and radiation barrier 112 is cut to desired dimensions. The edges 113 of the nylon sheets overhang the edges of the radiation barrier 111. The overhanging nylon sheets are located together and radio-frequency welded to complete the smart cap 110.

FIG. 12 shows personal protective radiation device in the form of a thyroid shield 120. The thyroid shield 120 includes a flexible internal radiation barrier 121 surrounded by a textile outer covering 122. The radiation barrier 121 is made from a sheet of a non-lead based, propriety material RadSafe Optima™. The outer covering 122 is made from two polyurethane sheets which have a thickness of 0.85 mm. The polyurethane sheets include quaternary ammonium compounds to provide an antimicrobial protective coating. The edges 123 of the outer covering 122 are radio-frequency welded together.

To produce the thyroid shield, the polyurethane sheets and radiation barrier is cut to desired dimensions. The edges of the polyurethane sheets overhang the edges of the radiation barrier. The overhanging polyurethane sheets are located together and radio-frequency welded to complete the thyroid shield.

FIG. 13 shows a personal protective radiation device in the form of an eye shield 130. The eye shield includes a flexible internal radiation barrier 131 surrounded by a textile outer covering 132. The radiation barrier 131 is made from a non-lead based, propriety material. The outer covering 132 is made from non-woven nylon having a denier of 100. The edges 133 of the outer covering are radio-frequency welded together.

To produce the eye shield 130, the nylon sheets and radiation barrier 132 is cut to desired dimensions. The edges 133 of the nylon sheets overhang the edges of the radiation barrier 132. The overhanging nylon sheets are located together and welded together using radio-frequency welding to complete the eye shield 130.

FIG. 14 shows personal protective radiation device in the form of a gonad/ovary shield 140. The gonad/ovary shield 140 includes a flexible internal radiation barrier 141 surrounded by a textile outer covering. The radiation barrier 141 is made from a sheet of a non-lead based, propriety material. The outer covering 142 is made from two woven polyethylene terephthalate which have a thickness of 0.3 mm. The edges 143 of the outer covering 142 are radio-frequency welded together.

To produce the gonad/ovary shield 140, the polyethylene terephthalate sheets and radiation barrier 141 is cut to desired dimensions. The edges 143 of the polyethylene terephthalate sheets overhang the edges of the radiation barrier 141. The overhanging polyethylene terephthalate sheets are located together and radio-frequency welded to complete the gonad shield 140.

FIGS. 15 shows personal protective radiation device in the form of shin guards 150. Each shin guard includes a flexible internal radiation barrier 151 surrounded by a textile outer covering 152. The radiation barrier 151 is made from a sheet of a non-lead based, propriety material RadSafe Optima™. The outer covering 152 is made from two, non-woven nylon sheets which each have a denier of 260. The nylon sheets include quaternary ammonium compounds to provide an antimicrobial protective coating. The edges 153 of the outer covering are heat welded together.

To produce each shin guards 150, the nylon sheets and radiation barrier 151 is cut to desired dimensions. The edges 153 of the nylon sheets overhang the edges of the radiation barrier 151. The overhanging nylon sheets are located together and heat welded together to complete the shin guards 150.

The personal protective radiation devices above all have weld joins at the edges. This produces personal protective radiation devices that have minimal microbial traps. Hence, infection control is substantially improved. It is also easy to clean and disinfect the personal protective radiation devices.

In this specification, terms such as upward, downward, horizontal and vertical, and their grammatical derivatives, are used to describe the invention in its normal orientation and are not to be construed to limit the invention to any particular orientation.

The reference to any prior art in this specification is not, and should not be taken as, an acknowledgement or any form of suggestion that the prior art forms part of the common general knowledge.

It should be appreciated that various other changes and modifications may be made to the embodiments described without departing from the spirit or scope of the invention.

What is claimed is:

1. A personal radiation protective device comprising:
a radiation barrier; and
an outer covering that covers the radiation barrier; the outer cover made from a material, wherein one or more edges of the outer covering are welded together.

2. The personal radiation protective device of claim 1 wherein the outer covering is made by two or more pieces of material.

3. The personal radiation protective device of claim 1 wherein any join on the outer covering which joins two or more pieces of material together is welded.

4. The personal radiation protective device of claim 1 wherein the radiation barrier is made from lead based material.

5. The personal radiation protective device of claim 1 wherein the radiation barrier is made from a non-lead based material.

6. The personal radiation protective device of claim 1 wherein the weld is a radio frequency weld, dielectric weld, high frequency weld, electrical impulse weld or heat weld.

7. The personal radiation protective device of claim 1 wherein the cover material is a textile or fabric.

8. The personal radiation protective device of claim 7 wherein the textile or fabric is a polymer or a plastic.

9. The personal radiation protective device of claim 1 wherein the outer covering includes an antimicrobial protective coating.

10. The personal radiation protective device of claim 1 wherein the personal radiation protective device includes a closure mechanism.

11. The personal radiation protective device of claim 10 wherein the closure mechanism is at least partially welded to the outer covering.

12. The personal radiation protective device of claim 10 wherein the closure mechanism is located inside the outer covering.

13. The personal radiation protective device of claim 10 wherein the closure mechanism includes one or more magnets.

14. The personal radiation protective device of claim 13 wherein the magnets are located within an over lapping portion of the outer covering.

15. The personal radiation protective device of claim 13 wherein the magnets are positioned laterally within the outer covering to provide closure for larger and smaller people.

16. The personal radiation protective device of claim 1 wherein indicia is located on the outer covering to indicate a safe level of overlap of the outer covering.

17. The personal radiation protective device of claim 1 wherein the personal radiation protective device includes a digital readable element.

18. The personal radiation protective device of claim 17 wherein the digital readable element is an RFID tag.

19. The personal radiation protective device of claim 17 wherein a digital readable element in the form of barcode tag is on the outer covering.

20. A method of producing a personal radiation protective device comprising the steps of:
cutting an outer covering to a desired dimension;
cutting or forming a radiation barrier to a desired dimension; and
welding the edges of the outer covering together.
